(19) United States
(12) Patent Application Publication
(10) Pub. No.: US 2009/0068138 A1
(43) Pub. Date: Mar. 12, 2009

(54) Polyglycerol anti-microbial agents and compositions

(76) Inventors: Xinyu Huang, Parsippany, NJ (US); Carmen Hendricks-Guy, White Plains, NY (US); Stewart Todd Elder, Butler, NJ (US); Andrea Preuss, Basel (CH); Ted Deisenroth, Brookfield, CT (US)

Correspondence Address: JoAnn Villamizar;Ciba Corporation/Patent Department, 540 White Plains Road, P.O. Box 2005, Tarrytown, NY, 10591, US

(21) Appl. No.: 12/283,067

(22) Filed: Sep. 9, 2008

Related U.S. Application Data
(60) Provisional application No. 60/993,259, filed on Sep. 11, 2007.
(60) Provisional application No. 61/062,633, filed on Jan. 28, 2008.

(51) Int. Cl. A61K 31/74; A61K 31/047; A61P 31/00; A61K 31/44

(52) U.S. Cl. 424/78.09; 514/738; 514/345

(57) ABSTRACT

Polyglycerol anti-microbial agents and compositions are provided. The agents are effective against a variety of pathogens including fungi, Gram positive bacteria and Gram negative bacteria yet are expected to have low human toxicity due in part to their polymeric nature. Applications for the polyglycerol anti-microbial agents and compositions include those involving human and plant contact, such as cosmetics, hair care products, textiles and plant protections, as well as in applications with much less human contact, such as plastics, coatings, wood, paper and other materials of construction.

This application claims benefit under 35 USC 119(e) of U.S. provisional application No. 60/993,259, filed Sep. 11, 2007 and 61/062,633, filed Jan. 28, 2008.

The preparation and use of compositions containing polyglycerol anti-microbial agents are provided. The agents are believed to have low human toxicity while being effective against a variety of pathogens and are useful in applications involving human contact, such as cosmetics, hair care products and textiles, as well as in applications with much less human contact, such as coatings.

Anti-microbial compounds are widely used and accepted as part of numerous products and materials. Anti-bacterial soaps, antifungal treatments for plants, topical medical treatments, anti-fouling coatings and disinfecting cleaners are just a few common uses of anti-microbial materials.

An apparent dilemma in the use of anti-microbial compounds is that such compounds must be active against living organisms but not be toxic toward humans, animals or desirable plants. Disclosed herein are compounds effective against a variety of harmful microbes expected to be less harmful to humans than many other anti-microbial compounds due in part to the polymeric nature of the compounds of the present invention.

U.S. Pat. Nos. 6,090,772; 5,955,408; 6,071,866; 6,358,906, incorporated herein in their entirety by reference, and WO96/06152 disclose compositions useful in personal care applications comprising triclosan as an anti-bacterial agent.

U.S. Pat. No. 5,635,462, incorporated herein in its entirety by reference, also discloses compositions comprising an anti-bacterial agent.

WO98/55096 discloses antimicrobial wipes having a porous sheet impregnated with an antibacterial composition containing an active antimicrobial agent.

U.S. Pat. No. 6,861,397, incorporated herein in its entirety by reference, discloses personal care and cleaning compositions having enhanced deposition of a topically active compound including antibacterial agents.

U.S. Pat. No. 6,872,241, incorporated herein in its entirety by reference, discloses anti-pathogenic air filtration media and air handling devices having protective capabilities against infectious airborne microorganisms.

US Published Pat. Appl. 20070265267, incorporated herein in its entirety by reference, discloses synergistic fungicidal compositions and a method of controlling phytopathogenic diseases on useful plants or on propagation material thereof, which comprises applying to the useful plants, the locus thereof or propagation material thereof the synergistic fungicidal composition.

Co pending U.S. patent application Ser. No. 11/656,863, incorporated herein in its entirety by reference, discloses substituted polyethylenimines effective as antimicrobial agents.

It is important that anti-microbial compounds, for example, as such as those found in antifungal and antibacterial compositions provide a substantial and broad spectrum reduction in microorganism populations quickly and without problems associated with toxicity and skin irritation.

The state of art for antimicrobial solution is the cocktail method, which provides a broad spectrum of antimicrobial activity by mixing two or more antimicrobial compounds. This method is usually associated with compatibility issues because of the difference of the physical and chemical properties of antimicrobial compounds, for example, different stability, solubility and leaching rate. One advantage of antimicrobial polymers is that a broad spectrum of antimicrobial activity can be achieved by combination of different functional groups onto the same polymer chain without generating any compatibility issues. Functional groups can also be introduced to tailor the physical and chemical properties of the antimicrobial polymers and therefore improve their performance in applications, for example, introducing appropriate functional groups onto the polymer chains can increase the solubility of the antimicrobial polymer in water and/or glycol without any influence on the antimicrobial activity.

Frey, et. al., Advanced Materials, vol 12, 2, 2000 p 235-239 discloses the preparation of hyperbranched polyether polyol polymers and copolymers from glycidol and/or glycidol derivatives such as allyl or phenyl glycidyl ether. Rokicki, et. al., Journal of Green Chemistry 2005, 7, p 529-539 disclose an alternate synthesis of the polymers starting from 4-(hydroxymethyl)-1,3-dioxalanone. The free hydroxyl groups of the polyols can be derivatized after polymerization via standard organic reactions.

It has been found that these hyperbranched polyether polyol polymers and copolymers and derivatives thereof are effective anti-microbial compounds against a wide spectrum of microbes including fungi, gram positive bacteria and gram negative bacteria. These polymers and co-polymers are quite effective against many common fungi such as those affecting human skin and scalp and many plants, for example, the polymers are effective anti-dandruff and plant protection agents.

### SUMMARY OF THE INVENTION

The present invention provides anti-microbial compositions comprising polyglycerol anti-microbial agents and methods for their use. Also disclosed are novel polyglycerol compounds and methods for their preparation. The polyglycerol anti-microbial agents are highly active against microbes upon contact, and remain active over a prolonged period of time due in part to their size and polymeric nature which makes them less susceptible to being unintentionally removed. They can be used to kill microbes on contact as in disinfection applications as well as preserve and protect materials against microbe infestation. The compounds are also expected to be less harmful upon human contact than other compounds that are more readily absorbed through the skin or made bio-available by dispersion into the environment. Such polyglycerols are hitherto unknown as anti-microbial agents.

The polyglycerol anti-microbial agents of the invention are polymers or co-polymers containing glycidyl repeat units. When referring to the polymers or copolymers herein, the all inclusive term “polymers” may be used to include both polymers and copolymers.

### DESCRIPTION OF THE INVENTION

The compositions of the of the present invention comprise polyglycerol anti-microbial agents which are hyperbranched polymers and dendrimers comprising in the backbone of the polymer glycerol derived moieties selected from

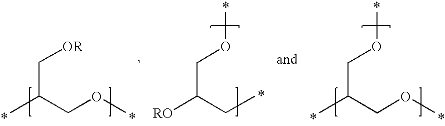

wherein R is independently H or a substituted or unsubstituted alkyl, alkenyl, alkyl carbonyl, alkenyl carbonyl, aryl or heterocycle which are incorporated into a home or personal care formulation, plant protection formulation, a natural or synthetic polymer, a coating or other material of construction.

For example, R is independently selected from H;

a) C₁₋₂₄ alkyl, C₃₋₂₄ alkenyl, C₁₋₂₄ alkylcarbonyl or C₃₋₂₄ alkenylcarbonyl which are uninterrupted or interrupted one or more times by one or more —O—, —N(R′)—, —CON(R′)—, —SO— or —SO₂—, and are unsubstituted or substituted one or more times by one or more C₃₋₆ cycloalkyl, —OR′, —COOR′, —COOM, —SO₃M, —SO₃H, phosphonic acid, halogen, —CONR′R′, —NR′R′, phosphonate salt, ammonium salt, group of the formulae

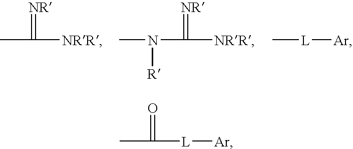

or group —Si(G)₃ wherein each G is independently hydroxyl, C₁₋₄ alkyl or C₁₋₄ alkoxy;
b) C₆₋₁₄ aromatic or C₁₋₉ saturated or unsaturated heterocycle which are unsubstituted or substituted one or more times by one or more groups R′, —OR′, —COOR′, —COOM, —SO₃M, —SO₃H, phosphonic acid, halogen, —CONR′R′, —NR′R′, phosphonate salt or ammonium salt, including a heterocycle of the formulae

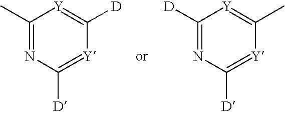

wherein Y and Y′ are independently N, C—R′, C—OR′ or C—NR′R′ and D and D′ are independently R′, —OR′ or —NR′R′; and
c) a group of the formulae

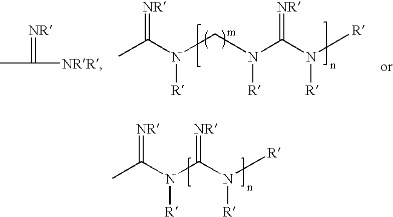

wherein m and n independently are a number from 1 to 12, preferably 1, 2, 3, 4, 5 or 6;
wherein each R′, independently of any other R′ is hydrogen;
a group

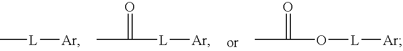

C₁₋₂₄ alkyl, C₃₋₂₄ alkenyl, C₃₋₆ cycloalkyl or C₁₋₂₄ alkylcarbonyl which are uninterrupted or interrupted one or more times by one or more oxygen atoms, sulfur atoms, carbonyl, —COO—, —CONH—, —NH—, —CON(C₁₋₂₄ alkyl)- or —N(C₁₋₂₄ alkyl)-,
which uninterrupted or interrupted alkyl, alkenyl, cycloalkyl or alkylcarbonyl are unsubstituted or substituted one or more times by one or more groups selected from halogen, —OH, C₂₋₂₄alkylcarbonyl, C₁₋₂₄alkoxy, C₂₋₂₄alkylcarboxy, —COOM, —CONH₂, —CON(H)(C₁₋₂₄alkyl), —CON(C₁₋₂₄alkyl)₂, —NH₂, —N(H)(C₁₋₂₄alkyl), —N(C₁₋₂₄alkyl)₂, —SO₃M, purine, pyridine, pyrimidine, triazine, imidazole, wherein each purine, pyridine, pyrimidine, triazine or imidazole is unsubstituted or substituted by one or more C₁₋₁₂ alkyl and wherein the purine, pyridine, pyrimidine, triazine or imidazole is neutral or ionically charged, phenyl, phenyl substituted one or more times by one or more C₁₋₈ alkyl, naphthyl, naphthyl substituted one or more times by one or more C₁₋₈ alkyl, amidine, guanidine, ammonium salt, phosphonic acid, phosphonate salt and a group

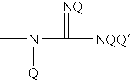

wherein each Q or Q′ is independently hydrogen, C₁₋₁₂alkyl, phenyl or benzyl;
or
when two R′ are attached to a nitrogen atom they may form, together with the nitrogen atom to which they are attached, form a 5-, 6- or 7-membered ring which is uninterrupted or interrupted by —O—, —NH— or —N(C₁₋₁₂ alkyl)-;
L is a direct bond, C₁₋₁₂ alkylene which is uninterrupted or interrupted by one or more oxygen atoms, —NH—, —N(C₁₋₁₂ alkyl) or phenylene and/or unsubstituted or substituted one or more times by one or more —OH, C₁₋₈ alkyl, C₁₋₂₄ alkoxy, C₂₋₂₄alkylcarboxy, —NH₂, —N(H)(C₁₋₈ alkyl), —N(C₁₋₈ alkyl)₂ or ammonium salt:
Ar is C₆₋₁₀ aromatic or C₁₋₉ saturated or unsaturated heterocycle which C₆₋₁₀ aromatic or C₁₋₉ saturated are unsubstituted or substituted one or more times by one or more halogen, —OH, C₁₋₂₄ alkoxy, C₂₋₂₄ alkylcarboxy, —COOQ″, —CONH₂, —CON(H)(C₁₋₈ alkyl), —CON(C₁₋₈ alkyl)₂, —NH₂, —N(H)(C₁₋₈ alkyl), —N(C₁₋₈ alkyl)₂, —SO₃M, SO₃H, ammonium salt, phosphonic acid, phosphonate salt, C₁₋₂₄ alkyl, C₁₋₂₄ alkyl or C₂₋₂₄ alkylcarboxy which is substituted one or more times by one or more groups selected from halogen, phenyl, phenyl substituted one or more times by one or more C₁₋₈ alkyl, naphthyl, purine, pyridine, pyrimidine, triazine and imidazole, wherein the purine, pyridine, pyrimidine, triazine or imidazole is unsubstituted or substituted by one or more C₁₋₁₂ alkyl and wherein the purine, pyridine, pyrimidine, triazine or imidazole is neutral or ionically charged;
wherein Q″ is hydrogen, C₁₋₂₄ alkyl, metal cation, ammonium salt, glycol ether, phenyl or benzyl, or phenyl or benzyl substituted one or more times by one or more halogen, hydroxy, C₁₋₂₄ alkoxy or C₁₋₁₂ alkyl,
M is a metal cation or an ammonium cation.

C₁₋₉ saturated or unsaturated heterocycle is a monocyclic or polycyclic ring of at least 3 atoms, containing 1-9 carbon atoms which heterocycle may also be ionically charged.

For example, C₁₋₉ saturated or unsaturated heterocycle is a 5, 6, or 7 membered ring containing 1, 2 or 3 nitrogen atoms which may be fused to another carbocylic or heterocyclic ring;

for example, C₁₋₉ saturated or unsaturated heterocycle is a 5, 6, or 7 membered ring containing 1, 2 or 3 nitrogen atoms which may be fused to a benzene ring;
for example, C₁₋₉ saturated or unsaturated heterocycle is a purine, imidazole, pyridine, pyramidine or triazole ring;
wherein the heterocyle may be substituted as described above and which heterocycle may also be ionically charged.

Alkyl is a straight or branched chain of the specified number of carbon atoms and is for example methyl, ethyl, n-propyl, n-butyl, sec-butyl, tert-butyl, n-hexyl, n-octyl, 2-ethylhexyl, n-nonyl, n-decyl, n-undecyl, n-dodecyl, n-tridecyl, n-tetradecyl, n-hexadecyl, n-octadecyl or docosanyl and the like.

Alkenyl is a straight or branched chain of the specified number of carbon atoms containing one or more carbon-carbon double bonds and is for example n-propenyl, n-butenyl, sec-butenyl, n-hexenyl, n-octenyl, n-hexadienyl, n-octadienyl, 2-ethylhexenyl, n-nonenyl, n-decenyl, n-undecenyl, n-dodecenyl, n-tridecenyl, n-tetradecenyl, n-hexadecenyl, n-octadecenyl, n-dodecadienyl, n-tetradecadienyl, n-hexadecadienyl, n-hexadecatrienyl, n-octadecadienyl, n-octadecatrienyl.

Alkyl carbonyl or alkanoyl is a straight or branched chain of the specified number of carbon atoms which has a carbonyl at the point of attachment.

An ammonium salt is, for example, unsubstituted ammonium, ammonium substituted 1, 2 or 3 times by one or more groups selected from

C₆₋₁₀aryl, C₁₋₂₄alkyl, C₁₋₂₄ branched alkyl, C₁₋₂₄alkyl and branched alkyl interrupted by one or more oxygen atoms, carbonyl, carboxy or C₆₋₁₀arylene,
and said aryl, alkyl, branched alkyl, interrupted alkyl and interrupted branched alkyl substituted by alkyl, aryl, OH, OAlkyl, OAcyl; plus a corresponding counter anion.

The ammonium salt may also comprise a ring or polycycle, which ring or polycycle may be substituted.

For example, the ammonium salt is tris benzyl ammonium or mono-, di-, or tri-C₁₋₂₄alkylammonium wherein each alkyl group can be the same or different, mono-, di-, or tri-benzyl, mono-, di-, or tri-C₁₋₂₄hydroxyalkylammonium wherein each alkyl group can be the same or different.

For example, the ammonium salt is di- or tri-substituted ammonium wherein each of the substituents are independently chosen from C₁₋₂₄alkyl, benzyl and C₁₋₂₄hydroxyalkyl.

The C₁₋₂₄alkyl, benzyl and C₁₋₂₄hydroxyalkyl groups of the substituted ammonium salts, may also be substituted by one or more C₁₋₂₄alkyl or branched alkyl, hydroxy, C₁₋₂₄carboxy ester, C₁₋₂₄alkyloxy, C₁₋₂₄acyloxy or halogen.

When M is an ammonium cation, it is for example, unsubstituted ammonium, ammonium substituted 1, 2, 3 or 4 times by one or more groups selected from C₁₋₂₄alkyl, C₁₋₂₄ branched alkyl, said alkyl and branched alkyl interrupted by one or more oxygen atoms, C₆₋₁₀aryl, C₇₋₉ aralkyl, and said alkyl, branched alkyl, interrupted alkyl and interrupted branched alkyl, and aryl substituted by alkyl, OH, OC₁₋₂₄alkyl, OC₁₋₂₄acyl.

The anti-microbial composition of the invention may comprise polymers wherein all glycidol derived moieties have the same R, e.g., all R groups are H or alkyl, but more generally, the polymers will comprise glycidol derived moieties wherein a portion or the groups R will be H and the remainder will be one or more groups described above. The groups R that are not H may be a single type of substituent, for example, a portion the groups R will be H and the remainder may be alkylcarbonyl; often, the remainder of the R groups which are not H will be a mixtures of the groups described above.

In many cases, the percentage of groups R which are hydrogen will be 90% or less, for example 80% or less, for example 50%, 25% or 10% or less.

For example, the polyglycerol anti-microbial agent is a polymer comprising a glycidol derived moiety wherein R is selected from H, C₁₋₂₄ alkyl, C₃₋₂₄ alkenyl, C₁₋₂₄ alkylcarbonyl and C₃₋₂₄ alkenylcarbonyl which are uninterrupted or interrupted one or more times by one or more —O—, —N(R′)—, —CON(R′)—, —SO— or —SO₂—, and are unsubstituted or substituted one or more times by one or more C₃₋₆ cycloalkyl, —OR′, —COOR′, —COOM, —SO₃M, —SO₃H, phosphonic acid, halogen, —CONR′R′, —NR′R′, phosphonate salt, ammonium salt, group of the formulae

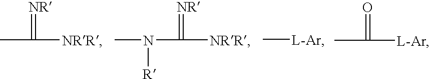

or group —Si(G)₃.

For example, the polyglycerol anti-microbial agent is a polymer which comprises a glycidol derived moiety wherein R is C₆₋₁₄ aromatic or C₁₋₉ saturated or unsaturated heterocycle which are unsubstituted or substituted one or more times by one or more groups R′, —OR′, —COOR′, —COOM, —SO₃M, —SO₃H, phosphonic acid, halogen, —CONR′R′, —NR′R′, phosphonate salt or ammonium salt.

For example, the polyglycerol anti-microbial agent is a polymer which comprises a glycidol derived moiety wherein R is a heterocycle of the formulae

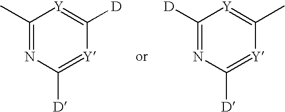

wherein Y and Y′ are independently N, C—R′, C—OR′ or C—NR′R′ and D and D′ are independently R′, —OR′ or —NR′R′.

For example, the polyglycerol anti-microbial agent is a polymer which comprises a glycidol derived moiety wherein R is a group of the formulae

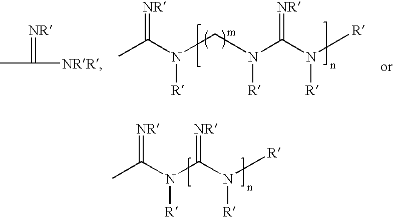

wherein m and n independently are 1, 2, 3, 4, 5 or 6.

For example, the polyglycerol anti-microbial agent is a polymer which comprises a glycidol derived moiety wherein R is C₁₋₂₄ alkyl or C₁₋₂₄ alkylcarbonyl which are uninterrupted or interrupted one or more times by —O—, —N(R′)—, —CON(R′)—, and are unsubstituted or substituted by one or more —NR′R′, halogen, ammonium salt, -L-Ar

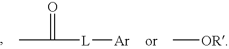

For example, R is selected from C₁₋₂₄ alkyl and C₁₋₂₄ alkylcarbonyl which are uninterrupted or interrupted one or more times by —O—, and substituted by one or more —NR′R′, halogen, ammonium salt, -L-Ar,

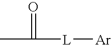

or —OR′, wherein R′ is hydrogen; -L-Ar,

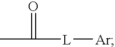

C₁₋₂₄ alkyl or C₁₋₂₄alkylcarbonyl which alkyl or alkylcarbonyl are uninterrupted or interrupted one or more times by one or more oxygen atoms, —COO—, —CONH—, —NH—, —CON(C₁₋₂₄ alkyl)- or —N(C₁₋₂₄ alkyl)- and which uninterrupted or interrupted alkyl or alkylcarbonyl are unsubstituted or substituted one or more times by one or more groups selected from halogen, —OH, C₂₋₂₄alkylcarbonyl, C₁₋₂₄alkoxy, C₂₋₂₄alkylcarboxy, —COOM, —CONH₂, —CON(H)(C₁₋₂₄alkyl), —CON(C₁₋₂₄alkyl)₂, —NH₂, —N(H)(C₁₋₂₄alkyl), —N(C₁₋₂₄alkyl)₂, purine, pyridine, pyrimidine, triazine, imidazole, wherein each purine, pyridine, pyrimidine, triazine or imidazole is unsubstituted or substituted by one or more C₁₋₁₂ alkyl and wherein the purine, pyridine, pyrimidine, triazine or imidazole is neutral or ionically charged, phenyl, phenyl substituted one or more times by one or more C₁₋₈ alkyl, naphthyl, naphthyl substituted one or more times by one or more C₁₋₈ alkyl and ammonium salt.

For example, an antimicrobial polyglycerol polymer or co-polymer which comprises a glycidol derived moiety wherein R is selected from C₁₋₂₄ alkyl, C₁₋₂₄ alkyl substituted one or more times by one or more NR′R′, halogen or ammonium salt; C₁₋₂₄ alkylcarbonyl, C₁₋₂₄ alkylcarbonyl substituted one or more times by one or more NR′R′, halogen or ammonium salt; benzyl, benzoyl which benzyl or benzoyl may be substituted one or more times by one or more halogens, hydroxyl, C₁₋₁₂ alkyl, C₁₋₁₂ alkoxy or C₁₋₁₂ alkylcarboxy; and C₁₋₂₄ alkyl or C₁₋₂₄ alkylcarbonyl substituted by

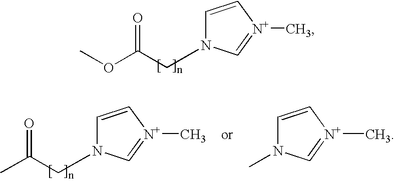

For example, the polyglycerol anti-microbial agent is a polymer which comprises a glycidol derived moiety wherein R is selected from

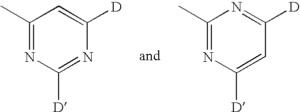

wherein D and D′ are independently R′, OR′ or NR′R′ wherein each R′ independently of any other R′ is hydrogen, ammonium salt, C₁₋₂₄ alkyl, C₁₋₂₄ alkanoyl which are unsubstituted or substituted one or more times by one or more halogen, hydroxyl or ammonium salt;

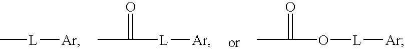

wherein L is a direct bond or C₁₋₁₂ alkylene and
Ar is phenyl or phenyl substituted one or more times by one or more halogen, —OH, C₁₋₂₄ alkoxy, C₂₋₂₄alkylcarboxy, —COOH, —COOM, —CONH₂, —CON(H)(C₁₋₁₂alkyl), —CON(C₁₋₁₂ alkyl)₂, —NH₂, —N(H)(C₁₋₂alkyl), —N(C₁₋₁₂alkyl)₂, ammonium salt, C₁₋₁₂ alkyl or alkyl substituted one or more times by one or more halogen.

For example, at least a portion of the groups R are selected from C₁₋₂₄ alkyl or C₁₋₂₄ alkylcarbonyl.

For example, the polyglycerol anti-microbial agent is a polymer which comprises a glycidol derived moiety wherein R is selected from C₁₋₂₄ alkyl or C₁₋₂₄ alkylcarbonyl which are substituted by at least one NR′R′ wherein each R′ is C₁₋₂₄ alkyl or C₁₋₂₄ alkylcarbonyl.

For example, the polyglycerol anti-microbial agent is a polymer which comprises a glycidol derived moiety wherein R is selected from C₁₋₂₄ alkyl or C₁₋₂₄ alkylcarbonyl which are substituted by at least one NR′R′ and at least one halogen, OR′SO₃M, SO₃H, or a group of the formulae L-Ar, or;

for example, C₁₋₂₄ alkyl or C₁₋₂₄ alkylcarbonyl substituted by at least one NR′R′ and at least one halogen, OR′ or a group of the formulae

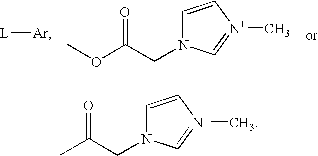

For example, R groups are selected from C₂₋₂₄ alkyl, C₂₋₂₄ alkylcarbonyl, C₃₋₂₄ alkenyl, and C₃₋₂₄ alkenylcarbonyl interrupted one or more times by one or more oxygen atoms, sulfur atoms, —SO— or —SO₂—, which are unsubstituted or substituted one or more times by one or more halogen, —OR′, —COOR′, —COOM, —CONR′R′, —NR′R′, —SO₃M, —SO₃H, phosphonic acid, phosphonate salt, ammonium salt or a group of the formulae

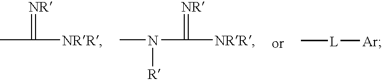

for example said interrupted alkyl or alkylcarbonyl, unsubstituted or substituted one or more times by one or more halogen, —OR′, —COOR, —COOM, CONR′R′, —NR′R′, ammonium salt or -L-Ar;
for example said interrupted alkyl or alkylcarbonyl, substituted one or more times by one or more halogen, —OR, CONR′R′, —NR′R′, ammonium salt or a group of the formulae

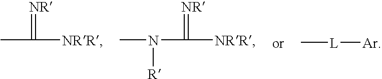

For example R may be selected from the group consisting of benzyl, benzyl substituted 1-5 times by F, Cl, Br or I or any combination of F, Cl, Br or I;

alkyl and alkanoyl substituted by pyramidine or triazine of the following formulae

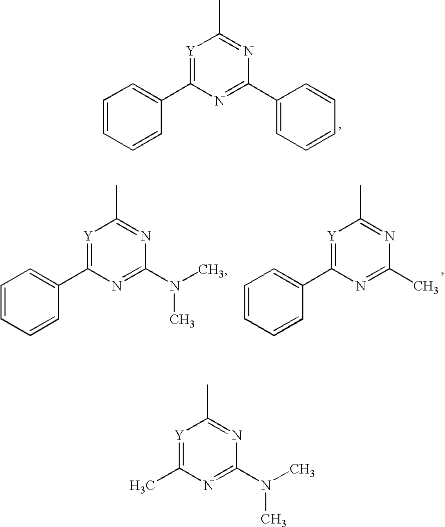

where Y is CR′ or N;
alkyl and alkylcarbonyl substituted by one or more NH₂,

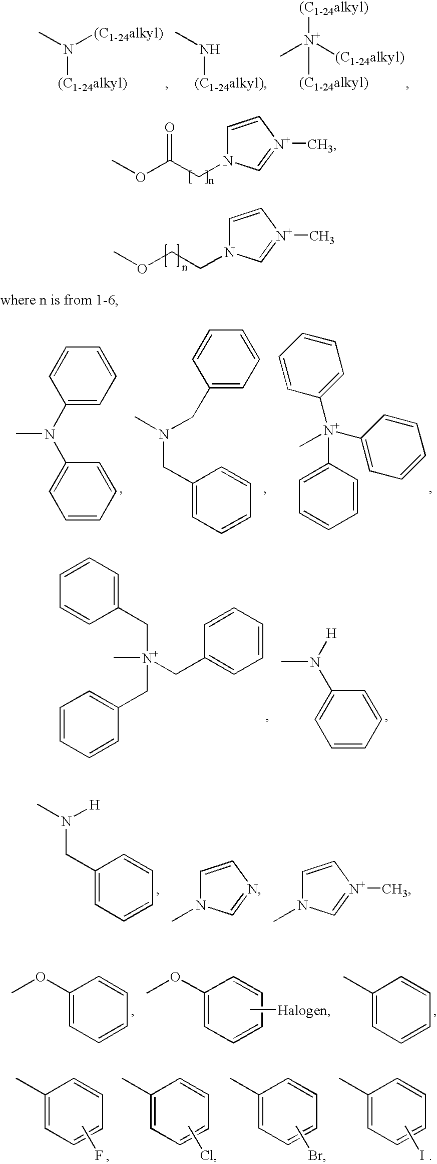

For example, R may be selected from the following formulae, isomers of the following formulae and homologues of said formulae and homologues of said isomers:

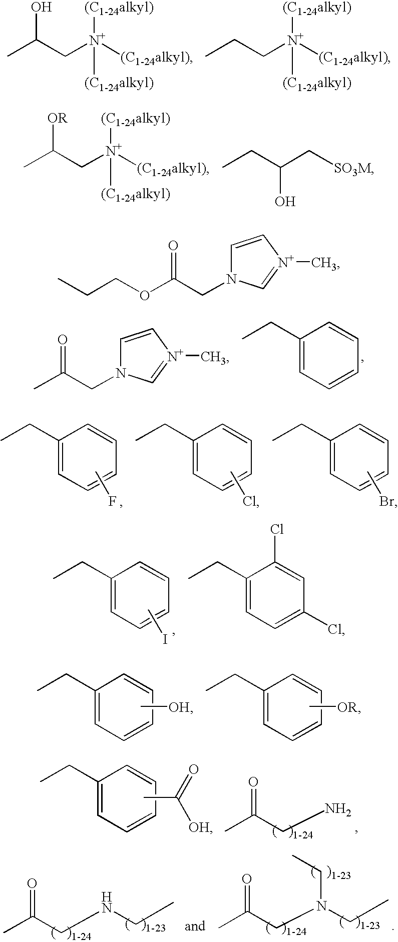

In one embodiment of the invention, the antimicrobial polymer comprises a glycidol derived moiety wherein R is an alkyl or alkyl carbonyl group which is substituted by at least two different groups selected from OR′, COOM, halogen, CONR′R′, NR′R′, SO₃M, SO₃H, phosphonic acid, phosphonate salt, ammonium salt or a group of the formulae

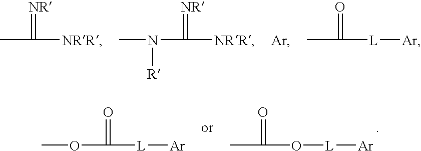

The polyglycerol anti-microbial agents may be substituted by moieties that provide different activities. For example, the polyglycerol polymer may bear substituents that render the polymer anti-bacterial and other substituents that render the polymer anti-fungal.

In one embodiment of the invention, a single polyglycerol anti-microbial polymer or co-polymer comprises at least two glycerol derived moieties with different groups R and in one embodiment the different groups provide different anti-microbial activity.

In another embodiment, a single R group can be multifunctional, for example, an alkyl group which alkyl group is substituted by two moieties, one moiety conferring anti-bacterial activity and another moiety conferring anti-fungal activity.

In another embodiment, at least two different inventive polyglycerol anti-microbial polymers or co-polymers are blended.

In another embodiment, an inventive polyglycerol anti-microbial polymer or co-polymer is blended with another anti-microbial compound.

These glycerol derived moieties are incorporated into the polymer backbone either via polymerization or copolymerization of a corresponding monomer, or by derivatizing a glycerol derived moiety wherein R is H after it has been incorporated into the polymer backbone through standard chemistry to introduce the selected R group.

Other groups may be included in the polymer backbone. For example, other monomers may be incorporated as a co monomer during polymerization, for example, copolymerization with an acrylate, styrene, vinyl alcohol etc. It is also possible that along with the glycerol derived moieties described herein, other glycerol derived moieties with alternate R groups may be present.

Many of the polymers or co-polymers of the anti-microbial compositions are prepared by the method of Frey, et. al., Advanced Materials, vol 12, 2, 2000 p 235-239 from glycidol, a glycidol ether, a mixture of glycidol and one or more glycidol ethers or mixture glycidol ethers and an initiator such as poly-hydroxy alcohols, amines, enamines, hydroxyalkyl amines which is therefore incorporated into the polymer. Other monomers may also be used in preparing copolymers of the invention, for example ethylene oxide, propylene oxide or other epoxy compounds.

Rokicki, et. al., Journal of Green Chemistry 2005, 7, p 529-539 disclose an alternate synthesis of the polymers starting from 4-(hydroxymethyl)-1,3-dioxalanone. Free hydroxy groups can be left as such or derivatized using known chemistry to generate for example, pendant ether, ester, carbonate, urea groups of the invention. Further modification of these introduced pendant groups may also be undertaken.

For example, hydroxy groups can be alkylated via reaction with alkyl halides, sulfonates, epoxides, etc. under the appropriate conditions, typically in the presence of a base. Alkylation also occurs via addition across a double bond as in reactions with vinyl esters, amides, nitriles sulphones etc. Hydroxyl groups can be acylated by reaction with acid halides, esters, anhydrides, carboxylic acids etc. A variety of metal catalyzed reactions, such as Heck and Suzuki reactions, are also known to derivatize amines.

The polymer or co-polymer prior has a molecular weight in the range of 300 to 50,000, for example 1,000 to 10,000.

Any number of process permutations can provide a wide variety of polyglycerol polymers and copolymers with varying R group substitution as described above. For example, polymerization of glycidol generates a branched polymer with a number of free hydroxyl groups. Copolymerization of glycidol with one or more glycidol ethers generates a branched polymer containing both free hydroxyl groups and pendant ether groups. A portion of the free hydroxyl groups from either of these polymers can then, for example be acylated with an acyl halide, or a mixture of acyl halides. Remaining free hydroxyls can then be acylated or alkylated by additional functionalization.

The reaction conditions will of course determine the amount of derivatized hydroxyl groups are formed. For example, when alkylating the hydroxyl group an alkyl mesylate, the amount of alkyl mesylate used in the reaction represents an upper limit of the amount of alkylating reagent that can be incorporated.

In addition to the glycerol derived moieties of the polymer backbone shown above and the optional presence of other co monomers or initiating agents, the polymer will also comprise end groups which are glycerol derived moieties such as

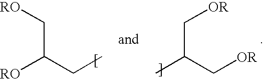

The polymers and co-polymers of the invention exhibit pronounced antimicrobial action, for example, against pathogenic gram-positive and gram-negative bacteria and against bacteria of the skin flora, and also against yeasts and molds. They are accordingly suitable for disinfection, deodorisation, and for general and antimicrobial treatment of the skin and mucosa and of integumentary appendages (hair), for example, for the disinfection of hands and wounds.

They are accordingly suitable as antimicrobial active substances and preservatives in personal care preparations, for example shampoos, bath additives, hair care preparations, liquid and solid soaps (based on synthetic surfactants and salts of saturated and/or unsaturated fatty acids), lotions and creams, deodorants, other aqueous or alcoholic solutions, e.g. cleansing solutions for the skin, moist cleaning cloths, oils or powders.

For example, the polyglycerol polymers and co-polymers of the invention are effective as anti-dandruff agents in shampoos.

The invention accordingly relates also to a personal care preparation comprising at least one antimicrobial polyglycerol polymer or co-polymer and cosmetically tolerable carriers or adjuvants.

The personal care preparation according to the invention contains from 0.01 to 15% by weight, for example, from 0.1 to 10% by weight, based on the total weight of the inventive composition, of the polymer or co-polymer, and cosmetically tolerable adjuvants.

Depending upon the form of the personal care preparation, it comprises, in addition to the antimicrobial polyglycerol polymer or co-polymer, further constituents, for example sequestering agents, colourings, perfume oils, thickening or solidifying agents (consistency regulators), emollients, UV-absorbers, skin protective agents, antioxidants, additives that improve the mechanical properties, such as dicarboxylic acids and/or aluminium, zinc, calcium or magnesium salts of C₁₄-C₂₂fatty acids, and, optionally, preservatives.

The personal care preparation according to the invention may be in the form of a water-in-oil or oil-in-water emulsion, an alcoholic or alcohol-containing formulation, a vesicular dispersion of an ionic or non-ionic ampiphilic lipid, a gel, a solid stick or an aerosol formulation.

As a water-in-oil or oil-in-water emulsion, the cosmetically tolerable adjuvant contains preferably from 5 to 50% of an oil phase, from 5 to 20% of an emulsifier and from 30 to 90% water. The oil phase may comprise any oil suitable for cosmetic formulations, for example one or more hydrocarbon oils, a wax, a natural oil, a silicone oil, a fatty acid ester or a fatty alcohol. Preferred mono- or poly-ols are ethanol, isopropanol, propylene glycol, hexylene glycol, glycerol and sorbitol.

Cosmetic formulations according to the invention are used in various fields. There come into consideration, for example, the following preparations:
skin-care preparations, e.g. skin-washing and cleansing preparations in the form of tablet-form or liquid soaps, synthetic detergents or washing pastes,
bath preparations, e.g. liquid (foam baths, milks, shower preparations) or solid bath preparations, e.g. bath cubes and bath salts;
skin-care preparations, e.g. skin emulsions, multi-emulsions or skin oils;
cosmetic personal care preparations, e.g. facial make-up in the form of day creams or powder creams, face powder (loose or pressed), rouge or cream make-up, eye-care preparations, e.g. eye shadow preparations, mascaras, eyeliners, eye creams or eye-fix creams; lip-care preparations, e.g. lipsticks, lip gloss, lip contour pencils, nail-care preparations, such as nail varnish, nail varnish removers, nail hardeners or cuticle removers;
intimate hygiene preparations, e.g. intimate washing lotions or intimate sprays;
foot-care preparations, e.g. foot baths, foot powders, foot creams or foot balsams, special deodorants and antiperspirants or callus-removing preparations;
light-protective preparations, such as sun milks, lotions, creams or oils, sun-blocks or tropicals, pre-tanning preparations or after-sun preparations;
skin-tanning preparations, e.g. self-tanning creams;
depigmenting preparations, e.g. preparations for bleaching the skin or skin-lightening preparations;
insect-repellents, e.g. insect-repellent oils, lotions, sprays or sticks;
deodorants, such as deodorant sprays, pump-action sprays, deodorant gels, sticks or roll-ons;
antiperspirants, e.g. antiperspirant sticks, creams or roll-ons;
preparations for cleansing and caring for blemished skin, e.g. synthetic detergents (solid or liquid), peeling or scrub preparations or peeling masks;
hair-removal preparations in chemical form (depilation), e.g. hair-removing powders, liquid hair-removing preparations, cream- or paste-form hair-removing preparations, hair-removing preparations in gel form or aerosol foams;
shaving preparations, e.g. shaving soap, foaming shaving creams, non-foaming shaving creams, foams and gels, preshave preparations for dry shaving, aftershaves or aftershave lotions;
fragrance preparations, e.g. fragrances (eau de Cologne, eau de toilette, eau de parfum, parfum de toilette, perfume), perfume oils or perfume creams;
dental care, denture-care and mouth-care preparations, e.g. toothpastes, gel toothpastes, tooth powders, mouthwash concentrates, anti-plaque mouthwashes, denture cleaners or denture fixatives;
cosmetic hair-treatment preparations, e.g. hair-washing preparations in the form of shampoos and conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, hair foams, hairsprays, bleaching preparations, e.g. hydrogen peroxide solutions, lightening shampoos, bleaching creams, bleaching powders, bleaching pastes or oils, temporary, semi-permanent or permanent hair colorants, preparations containing self-oxidising dyes, or natural hair colorants, such as henna or camomile.

The following represent examples of various formulations containing the antimicrobial polyglycerol of the invention. Obviously, these are simple, basic formulations only and a wide variety of similar formulations are known in the art into which the present antimicrobial polyglycerols at various concentrations are readily incorporated.

An antimicrobial soap has, for example, the following composition:

0.01 to 5% by weight of antimicrobial polyglycerol polymer or co-polymer,
0.3 to 1% by weight titanium dioxide,
1 to 10% by weight stearic acid,
   soap base ad 100%, e.g. a sodium salt of tallow fatty acid or coconut fatty acid, or glycerol.

A shampoo has, for example, the following composition:

0.01 to 5% by weight of antimicrobial polyglycerol polymer or co-polymer,
12.0% by weight sodium laureth-2-sulfate,
4.0% by weight cocamidopropyl betaine,
3.0% by weight NaCl and
water ad 100%.

A deodorant has, for example, the following composition:

0.01 to 5% by weight antimicrobial polyglycerol polymer or co-polymer,
60% by weight ethanol,
0.3% by weight perfume oil, and
water ad 100%.

The invention relates also to an oral composition containing from 0.01 to 15% by weight, based on the total weight of the composition, of the antimicrobial polyglycerol polymer or co-polymer, and orally tolerable adjuvants.

Example of an oral composition:

10% by weight sorbitol,
10% by weight glycerol,
15% by weight ethanol,
15% by weight propylene glycol,
0.5% by weight sodium lauryl sulfate,
0.25% by weight sodium methylcocyl taurate,
0.25% by weight polyoxypropylene/polyoxyethylene block copolymer,
0.10% by weight peppermint flavouring,
0.1 to 0.5% by weight of antimicrobial polyglycerol polymer or co-polymer, and
48.6% by weight water.

The oral composition according to the invention may be, for example, in the form of a gel, a paste, a cream or an aqueous preparation (mouthwash).

The oral composition according to the invention may also comprise compounds that release fluoride ions which are effective against the formation of caries, for example inorganic fluoride salts, e.g. sodium, potassium, ammonium or calcium fluoride, or organic fluoride salts, e.g. amine fluorides, which are known under the trade name OLAFLUOR.

The antimicrobial polyglycerol polymers or co-polymers of this invention are also suitable for treating, especially preserving, textile fibre materials. Such materials are undyed and dyed or printed fibre materials, e.g. of silk, wool, polyamide or polyurethanes, and especially cellulosic fibre materials of all kinds. Such fibre materials are, for example, natural cellulose fibres, such as cotton, linen, jute and hemp, as well as cellulose and regenerated cellulose.

The antimicrobial polyglycerol polymers or co-polymers of this invention are suitable also for treating, especially imparting antimicrobial properties to or preserving, plastics, e.g. polyethylene, polypropylene, polyurethane, polyester, polyamide, polycarbonate, latex etc. Fields of use therefore are, for example, floor coverings, plastics coatings, plastics containers and packaging materials; kitchen and bathroom utensils (e.g. brushes, shower curtains, sponges, bathmats), latex, filter materials (air and water filters), plastics articles used in the field of medicine, e.g. dressing materials, syringes, catheters etc., so-called “medical devices”, gloves and mattresses.

The antimicrobial polyglycerol polymers or co-polymers of this invention are suitable also for treating, especially imparting antimicrobial properties to or preserving industrial formulations such as coatings, lubricants etc.

Paper, for example papers used for hygiene purposes, may also be provided with antimi-crobial properties using the polyglycerol polymers or co-polymers of this invention.

It is also possible for nonwovens, e.g. nappies/diapers, sanitary towels, panty liners, and cloths for hygiene and household uses, to be provided with antimicrobial properties in accordance with the invention.

The antimicrobial polyglycerol polymers or co-polymers of this invention are also used in washing and cleaning formulations, e.g. in liquid or powder washing agents or softeners.

The antimicrobial polyglycerol polymers or co-polymers can also be used in household and general-purpose cleaners for cleaning and disinfecting hard surfaces.

A cleaning preparation has, for example the following composition:

0.01 to 5% by weight antimicrobial polyglycerol polymer or co-polymer
3.0% by weight octyl alcohol 4EO
1.3% by weight fatty alcohol C₈-C₁₀polyglucoside
3.0% by weight isopropanol
water ad 100%.

In addition to preserving cosmetic and household products, the preservation of technical products, the provision of technical products with antimicrobial properties and use as a biocide in technical processes are also possible, for example in paper treatment, especially in paper treatment liquors, printing thickeners of starch or cellulose derivatives, surface-coatings and paints.

The antimicrobial polyglycerol polymers or co-polymers of the invention are also suitable for the antimicrobial treatment of wood and for the antimicrobial treatment of leather, the preserving of leather and the provision of leather with antimicrobial properties.

The compounds according to the invention are also suitable for the protection of cosmetic products and household products from microbial damage.

Co-pending application 60/720,662, which is hereby incorporated in its entirety by reference, discloses compounds useful in coatings or films in protecting surfaces from bio-fouling. Such surfaces include surfaces in contact with marine environments (including fresh water, brackish water and salt water environments), for example, the hulls of ships, surfaces of docks or the inside of pipes in circulating or pass-through water systems. Other surfaces are susceptible to similar biofouling, for example walls exposed to rain water, walls of showers, roofs, gutters, pool areas, saunas, floors and walls exposed to damp environs such as basements or garages and even the housing of tools and outdoor furniture.

The antimicrobial polyglycerol polymers or co-polymers of this invention are also useful in preventing bio-fouling, or eliminating or controlling microbe accumulation on surfaces described in co-pending application 60/720,662 either by incorporating the antimicrobial ethylenimine polymers or co-polymers into the article or surface of the article in question or by applying the antimicrobial ethylenimine polymers or co-polymers to these surfaces either directly or as part of a coating or film as described in the co-pending application.

When applied as a part of a film or coating, the antimicrobial polyglycerol polymers or co-polymers of this invention are part of a composition which also comprises a binder.

The binder may be any polymer or oligomer compatible with the present antimicrobials. The binder may be in the form of a polymer or oligomer prior to preparation of the anti-fouling composition, or may form by polymerization during or after preparation, including after application to the substrate. In certain applications, such as certain coating applications, it will be desirable to crosslink the oligomer or polymer of the anti fouling composition after application.

The term binder as used in the present invention also includes materials such as glycols, oils, waxes and surfactants commercially used in the care of wood, plastic, glass and other surfaces. Examples include water proofing materials for wood, vinyl protectants, protective waxes and the like.

The composition may be a coating or a film. When the composition is a thermoplastic film which is applied to a surface, for example, by the use of an adhesive or by melt applications including calendaring and co-extrusion, the binder is the thermoplastic polymer matrix used to prepare the film.

When the composition is a coating, it may be applied as a liquid solution or suspension, a paste, gel, oil or the coating composition may be a solid, for example a powder coating which is subsequently cured by heat, UV light or other method.

As the composition of the invention may be a coating or a film, the binder can be comprised of any polymer used in coating formulations or film preparation. For example, the binder is a thermoset, thermoplastic, elastomeric, inherently crosslinked or crosslinked polymer.

Thermoset, thermoplastic, elastomeric, inherently crosslinked or crosslinked polymers include polyolefin, polyamide, polyurethane, polyacrylate, polyacrylamide, polycarbonate, polystyrene, polyvinyl acetates, polyvinyl alcohols, polyester, halogenated vinyl polymers such as PVC, natural and synthetic rubbers, alkyd resins, epoxy resins, unsaturated polyesters, unsaturated polyamides, polyimides, silicon containing and carbamate polymers, fluorinated polymers, crosslinkable acrylic resins derived from substituted acrylic esters, e.g. from epoxy acrylates, urethane acrylates or polyester acrylates. The polymers may also be blends and copolymers of the preceding chemistries.

Biocompatible coating polymers, such as, poly[-alkoxyalkanoate-co-3-hydroxyalkenoate] (PHAE) polyesters, Geiger et. al. Polymer Bulletin 52, 65-70 (2004), can also serve as binders in the present invention.

Alkyd resins, polyesters, polyurethanes, epoxy resins, silicone containing polymers, fluorinated polymers and polymers of vinyl acetate, vinyl alcohol and vinyl amine are non-limiting examples of common coating binders useful in the present invention. Other coating binders, of course, are part of the present invention.

Coatings are frequently crosslinked with, for example, melamine resins, urea resins, isocyanates, isocyanurates, polyisocyanates, epoxy resins, anhydrides, poly acids and amines, with or without accelerators.

The compositions of present invention are for example a coating applied to a surface which is exposed to conditions favorable for bioaccumulation. The presence of the antimicrobial ethylenimine polymers or co-polymers of this invention in said coating will prevent the adherence of organisms to the surface.

The anti-microbial polymer or copolymers of the present invention may be part of a complete coating or paint formulation, such as a marine gel-coat, shellac, varnish, lacquer or paint, or the anti fouling composition may comprise only a polymer of the instant invention and binder, or a polymer of the instant invention, binder and a carrier substance. It is anticipated that other additives encountered in such coating formulations or applications will find optional use in the present applications as well.

The coating may be solvent borne or aqueous. Aqueous coatings are typically considered more environmentally friendly.

The coating is, for example, aqueous dispersion of a polymer of the instant invention and a binder or a water based coating or paint. For example, the coating comprises an aqueous dispersion of a polymer of the instant invention and an acrylic, methacrylic or acrylamide polymers or co-polymers or a poly[-alkoxyalkanoate-co-3-hydroxyalkenoate] polyester.

The coating is, for example, a coating or varnish used in marine applications.

The coating may be applied to a surface which has already been coated, such as a protective coating, a clear coat or a protective wax applied over a previously coated article.

Coating systems include marine coatings, wood coatings, other coatings for metals and coatings over plastics and ceramics. Exemplary of marine coatings are gel coats comprising an unsaturated polyester, a styrene and a catalyst.

The coating is, for example a house paint, or other decorative or protective paint. It may be a paint or other coating that is applied to cement, concrete or other masonry article. The coating may be a water proofer as for a basement or foundation.

The coating composition is applied to a surface by any conventional means including spin coating, dip coating, spray coating, draw down, or by brush, roller or other applicator. A drying or curing period will typically be needed.

Coating or film thickness will vary depending on application and will become apparent to one skilled in the art after limited testing.

The composition may be in the form of a protective laminate film.

Such a film typically comprises thermoset, thermoplastic, elastomeric, or crosslinked polymers. Examples of such polymers include, but are not limited to, polyolefin, polyamide, polyurethane, polyacrylate, polyacrylamide, polycarbonate, polystyrene, polyvinyl acetates, polyvinyl alcohols, polyester, halogenated vinyl polymers such as PVC, natural and synthetic rubbers, alkyd resins, epoxy resins, unsaturated polyesters, unsaturated polyamides, polyimides, fluorinated polymers, silicon containing and carbamate polymers. The polymers may also be blends and copolymers of the preceding chemistries.

When the anti-fouling composition is a preformed film it is applied to the surface by, for example, the use of an adhesive, or co-extruded onto the surface. It may also be mechanically affixed via fasteners which may require the use of a sealant or caulk wherein the esters of the instant invention may also be advantageously employed.

A plastic film may also be applied with heat which includes calendaring, melt applications and shrink wrapping.

The composition may be part of a polish, such a furniture polish, or a dispersant or surfactant formulation such as a glycol or mineral oil dispersion or other formulation as used in for example wood protection.

Examples of useful surfactants include, but are not limited to, polyoxyethylene-based surface-active substances, including polyoxyethylene sorbitan tetraoleate (PST), polyoxyethylene sorbitol hexaoleate (PSH), polyoxyethylene 6 tridecyl ether, polyoxyethylene 12 tridecyl ether, polyoxyethylene 18 tridecyl ether, TWEEN® surfactants, TRITON® surfactants, and the polyoxyethlene-polyoxypropylene copolymers such as the PLURONIC® and POLOXAMER® product series (from BASF). Other matrix-forming components include dextrans, linear PEG molecules (MW 500 to 5,000,000), star-shaped PEG molecules, comb-shaped and dendrimeric, hyperbrached PEG molecules, as well as the analogous linear, star, and dendrimer polyamine polymers, and various carbonated, perfluorinated (e.g., DUPONT ZONYL® fluorosurfactants) and siliconated (e.g., dimethylsiloxane-ethylene oxide block copolymers) surfactants.

Given the wide array of applications for the present anti-microbial compositions, the composition may contain other additives such as antioxidants, UV absorbers, hindered amines, phosphites or phosphonites, benzofuran-2-ones, thiosynergists, polyamide stabilizers, metal stearates, nucleating agents, fillers, reinforcing agents, lubricants, emulsifiers, dyes, pigments, dispersants, other optical brighteners, flame retardants, antistatic agents, blowing agents and the like, such as the materials listed below, or mixtures thereof.

The substrate can be an inorganic or organic substrate, for example, a metal or metal alloy; a thermoplastic, elastomeric, inherently crosslinked or crosslinked polymer as described above; a natural polymer such as wood or rubber; a ceramic material; glass; leather or other textile.

The substrate may be, for example, non-metal inorganic surfaces such as silica, silicon dioxide, titanium oxides, aluminum oxides, iron oxides, carbon, silicon, various silicates and sol-gels, masonry, and composite materials such as fiberglass and plastic lumber (a blend of polymers and wood shavings, wood flour or other wood particles).

The inorganic or organic substrate is, for example, a metal or metal alloy, a thermoplastic, elastomeric, inherently crosslinked or crosslinked polymer, a ceramic material or a glass.

The substrate may be a multi-layered article comprised of the same or different components in each layer. The surface coated or laminated may be the exposed surface of an already applied coating or laminate.

The inorganic or organic substrate to be coated or laminated can be in any solid form. For example, polymer substrates may be plastics in the form of films, injection-molded articles, extruded workpieces, fibres, felts or woven fabrics.

For example molded or extruded polymeric articles used in construction or the manufacture of durable goods such as siding, fascia and mailboxes can all benefit from the present method for stabilizer replenishment.

Plastics which would benefit from the present method include, but are not limited to, plastics used in construction or the manufacture of durable goods or machine parts, including outdoor furniture, boats, siding, roofing, glazing, protective films, decals, sealants, composites like plastic lumber and fiber reinforced composites, functional films including films used in displays as well as articles constructed from synthetic fibers such as awnings, fabrics such as used in canvas or sails and rubber articles such as outdoor matting and other uses cited in this disclosure. Examples include polypropylene, polyethylene, PVC, POM, polysulfones, styrenics, polyamides, urethanes, polyesters, polycarbonate, acrylics, butadiene, thermoplastic polyolefins, ionomers, unsaturated polyesters and blends of polymer resins including ABS, SAN and PC/ABS.

The polyglycerol polymers and co-polymers of the invention are also effective in protecting useful plants, such as plants in agriculture, in horticulture and in forests, plant parts and seeds from disease and spoilage. For example, the present invention also provides a method which comprises applying to useful plants, the locus thereof or propagation material thereof a composition which comprises at least one of the polyglycerol polymers and co-polymers of the invention. Said compositions can be used as foliar, soil and seed treatment fungicides.

The compositions of the invention it is possible to inhibit or destroy the phytopathogenic microorganisms which occur in plants or in parts of plants (fruit, blossoms, leaves, stems, tubers, roots) in different useful plants. The present compositions are applied by treating the fungi, the useful plants, the locus thereof, the propagation material thereof, the natural substances of plant origin, which have been taken from the natural life cycle, and/or their processed forms, or the industrial materials threatened by fungus attack with the compositions in an effective amount.

The compositions according to the invention may be applied before or after infection of the useful plants, the propagation material thereof, the natural substances of plant and/or animal origin, which have been taken from the natural life cycle, and/or their processed forms, or the industrial materials by the fungi.

The compositions of the present invention are of particular interest for controlling a large number of fungi in various useful plants or their seeds, especially in field crops such as potatoes, tobacco and sugar beets, and wheat, rye, barley, oats, rice, maize, lawns, cotton, soybeans, oil seed rape, pulse crops, sunflower, coffee, sugarcane, fruit and ornamentals in horticulture and viticulture, in vegetables such as cucumbers, beans and cucurbits.

When applied to plants, the polyglycerol polymers and co-polymers of the invention are applied at a rate of 1 to 5000 g a.i./ha, for example 2 to 2000 g a.i./ha, for example, 5 to 2000 g a.i./ha, for example, 10 to 1000 g a.i./ha, e.g. 50, 75, 100, 200, 250, 500, 800, 1000, 1500 g a.i./ha of polymer or co-polymers.

In agricultural practice the application rates depend on the type of effect desired, and typically range from 20 to 4000 g of total antimicrobials per hectare.

When treating seed, rates of 0.001 to 50 g of the present polyglycerol polymers and co-polymers, for example 0.01 to 10 g, per kg of seed, are generally sufficient.

The composition comprising the polyglycerol polymers and co-polymers of the invention may be employed in any conventional form, for example in the form a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate formulation inerts (diluents, solvents, fillers and optionally other formulating ingredients such as surfactants, biocides, anti-freeze, stickers, thickeners and compounds that provide adjuvancy effects). For example, formulations to be applied in spraying forms, such as water dispersible concentrates (e.g. EC, SC, DC, OD, SE, EW, EO and the like), wettable powders and granules, typically contain surfactants such as wetting and dispersing agents and other compounds that provide adjuvancy effects.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of at least one of the polyglycerol polymers and co-polymers, from 0 to 20% agriculturally acceptable surfactant and 10 to 99.99% solid or liquid formulation inerts and adjuvant(s), and optionally other active agents, particularly microbiocides or conservatives or the like.

Concentrated forms of compositions generally contain in between about 2 and 80%, for example, between about 5 and 70% by weight of total active agent. Application forms of formulation may for example contain from 0.01 to 20% by weight, for example from 0.01 to 5% by weight of active agent.

Methods of preparing the above plant protection formulations are well known, for example, in US Published Pat. Appl. 20070265267, already incorporated by reference.

Particular embodiments of the invention therefore relate to

methods for protecting plastics, coatings, other materials of construction, home or personal care formulations, plants, agricultural products, industrial formulations or technical process against the action of microbes which comprises adding an effective amount of the present polymer or copolymer to the formulation or process;
a method for protecting skin, mucosa and integumentary appendages against the action of microbes including protecting the scalp from dandruff, which comprises applying a preparation comprising an effective amount of the present polymer or copolymer;
a method for protecting paper, wood, leather, synthetic textile materials or natural textile materials such as cotton against the action of microbes comprising incorporating or applying an effective amount of the present polymer or copolymer or a composition comprising an effective amount the present polymer or copolymer;
a method for cleaning and disinfecting hard surfaces which comprises applying a preparation comprising an effective amount of the present polymer or copolymer;
a method for preventing bio-fouling of an article comprising incorporating the present antimicrobial polymer or co-polymer into the article or surface of the article or by applying the antimicrobial ethylenimine polymer or co-polymer to these surfaces either directly or as part of a coating or film.

Other materials of construction include, in addition to wood, metals, paper, glass, ceramics, coatings, plastics and textiles, materials such as concrete, cement, adhesives, caulking materials, composites of natural and synthetic materials etc.

While some of the polyglycerol anti-microbial agents of the inventive compositions are known compounds, many are novel. The novel polymers are prepared from a combination of the above described reactions combined with standard derivation reactions. For example, novel compounds include hyperbranched polymers and dendrimers comprising in the backbone of the polymer the glycerol derived moieties

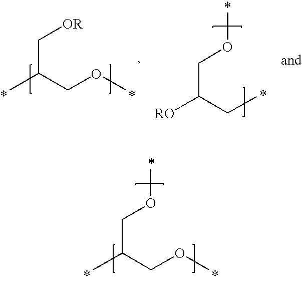

wherein the groups R are selected from
a) C₁₋₂₄ alkyl, C₃₋₂₄ alkenyl, C₁₋₂₄ alkylcarbonyl or C₃₋₂₄ alkenylcarbonyl which are interrupted one or more times by one or more —O—, —N(R′)—, —CON(R′)—, —SO— or —SO₂—, and/or substituted one or more times by one or more —OR′, —CONR′R′, —NR′R′, ammonium salt, group of the formulae

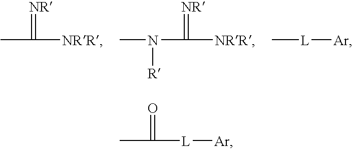

or group —Si(G)₃ wherein each G is independently hydroxyl, C₁₋₄ alkyl or C₁₋₄ alkoxy;
b) C₁₋₉ saturated or unsaturated heterocycle which are unsubstituted or substituted one or more times by one or more groups R′, —OR′, —COOR′, —COOM, —SO₃M, —SO₃H, phosphonic acid, halogen, —CONR′R′, —NR′R′, phosphonate salt or ammonium salt, including a heterocycle of the formulae

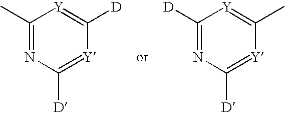

wherein Y and Y′ are independently N, C—R′, C—OR′ or C—NR′R′ and D and D′ are independently R′, —OR′ or —NR′R′; and
c) group of the formulae

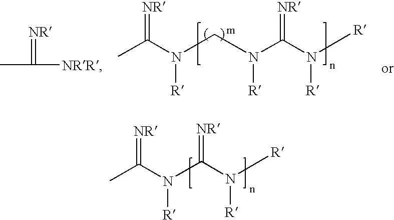

wherein m and n independently are a number from 1 to 12, preferably 1, 2, 3, 4, 5 or 6;
wherein each R′, independently of any other R′ is hydrogen;
a group

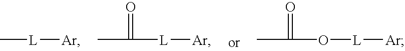

C₁₋₂₄ alkyl, C₃₋₂₄ alkenyl, C₃₋₆ cycloalkyl or C₁₋₂₄ alkylcarbonyl which are uninterrupted or interrupted one or more times by one or more oxygen atoms, sulfur atoms, carbonyl, —COO—, —CONH—, —NH—, —CON(C₁₋₂₄alkyl)- or —N(C₁₋₂₄alkyl)-,
which uninterrupted or interrupted alkyl, alkenyl, cycloalkyl or alkylcarbonyl are unsubstituted or substituted one or more times by one or more groups selected from halogen, —OH, C₂₋₂₄alkylcarbonyl, C₁₋₂₄alkoxy, C₂₋₂₄alkylcarboxy, —COOM, —CONH₂, —CON(H)(C₁₋₂₄ alkyl), —CON(C₁₋₂₄ alkyl)₂, —NH₂, —N(H)(C₁₋₂₄ alkyl), —N(C₁₋₂₄ alkyl)₂, —SO₃M, purine, pyridine, pyrimidine, triazine, imidazole, wherein each purine, pyridine, pyrimidine, triazine or imidazole is unsubstituted or substituted by one or more C₁₋₁₂ alkyl and wherein the purine, pyridine, pyrimidine, triazine or imidazole is neutral or ionically charged, phenyl, phenyl substituted one or more times by one or more C₁₋₈ alkyl, naphthyl, naphthyl substituted one or more times by one or more C₁₋₈ alkyl, amidine, guanidine, ammonium salt, phosphonic acid, phosphonate salt and a group

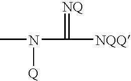

wherein each Q or Q′ is independently hydrogen, C₁₋₁₂alkyl, phenyl or benzyl;
or
when two R′ are attached to a nitrogen atom they may form, together with the nitrogen atom to which they are attached, form a 5-, 6- or 7-membered ring which is uninterrupted or interrupted by —O—, —NH— or —N(C₁₋₁₂ alkyl)-;
L is a direct bond, C₁₋₁₂ alkylene which is uninterrupted or interrupted by one or more oxygen atoms, —NH—, —N(C₁₋₁₂ alkyl) or phenylene and/or unsubstituted or substituted one or more times by one or more —OH, C₁₋₈ alkyl, C₁₋₂₄ alkoxy, C₂₋₂₄alkylcarboxy, —NH₂, —N(H)(C₁₋₈ alkyl), —N(C₁₋₈ alkyl)₂ or ammonium salt:
Ar is C₆₋₁₀ aromatic or C₁₋₉ saturated or unsaturated heterocycle which C₆₋₁₀ aromatic or C₁₋₉ saturated are unsubstituted or substituted one or more times by one or more halogen, —OH, C₁₋₂₄ alkoxy, C₂₋₂₄alkylcarboxy, —COOQ″, —CONH₂, —CON(H)(C₁₋₈ alkyl), —CON(C₁₋₈ alkyl)₂, —NH₂, —N(H)(C₁₋₈ alkyl), —N(C₁₋₈ alkyl)₂, —SO₃M, SO₃H, ammonium salt, phosphonic acid, phosphonate salt, C₁₋₂₄ alkyl, C₁₋₂₄ alkyl or C₂₋₂₄ alkylcarboxy which is substituted one or more times by one or more groups selected from halogen, phenyl, phenyl substituted one or more times by one or more C₁₋₈ alkyl, naphthyl, purine, pyridine, pyrimidine, triazine and imidazole, wherein the purine, pyridine, pyrimidine, triazine or imidazole is unsubstituted or substituted by one or more C₁₋₁₂ alkyl and wherein the purine, pyridine, pyrimidine, triazine or imidazole is neutral or ionically charged;
wherein Q″ is hydrogen, C₁₋₂₄ alkyl, metal cation, ammonium salt, glycol ether, phenyl or benzyl, or phenyl or benzyl substituted one or more times by one or more halogen, hydroxy, C₁₋₂₄ alkoxy or C₁₋₁₂ alkyl,
M is a metal cation or an ammonium cation.

For example, the polyglycerol polymer comprising at least one moiety of the above formulae wherein R is selected from C₁₋₂₄ alkyl or C₁₋₂₄ alkylcarbonyl which are interrupted one or more times by —O—, —N(R′)—, —CON(R′)—, and/or substituted by one or more —NR′R′, halogen, ammonium salt, -L-Ar,

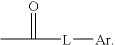

For example at least a portion of the groups R are C₁₋₂₄ alkyl or C₁₋₂₄ alkylcarbonyl which interrupted one or more times by —O—, and substituted by one or more —NR′R′, halogen, ammonium salt, -L-Ar,

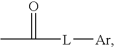

wherein
R′ is hydrogen; -L-Ar,

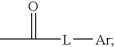

C₁₋₂₄ alkyl or C₁₋₂₄ alkylcarbonyl which alkyl or alkylcarbonyl are uninterrupted or interrupted one or more times by one or more oxygen atoms, —COO—, —CONH—, —NH—, —CON(C₁₋₂₄ alkyl)- or —N(C₁₋₂₄ alkyl)- and which uninterrupted or interrupted alkyl or alkylcarbonyl are unsubstituted or substituted one or more times by one or more groups selected from halogen, —OH, C₂₋₂₄alkylcarbonyl, C₁₋₂₄alkoxy, C₂₋₂₄alkylcarboxy, —COOM, —CONH₂, —CON(H)(C₁₋₂₄ alkyl), —CON(C₁₋₂₄ alkyl)₂, —NH₂, —N(H)(C₁₋₂₄alkyl), —N(C₁₋₂₄alkyl)₂, purine, pyridine, pyrimidine, triazine, imidazole, wherein each purine, pyridine, pyrimidine, triazine or imidazole is unsubstituted or substituted by one or more C₁₋₁₂ alkyl and wherein the purine, pyridine, pyrimidine, triazine or imidazole is neutral or ionically charged, phenyl, phenyl substituted one or more times by one or more C₁₋₈ alkyl, naphthyl, naphthyl substituted one or more times by one or more C₁₋₈ alkyl and ammonium salt.

For example, R is selected from C₁₋₂₄ alkyl or C₁₋₂₄ alkyl substituted one or more times by one or more NR′R′, halogen or ammonium salt; C₁₋₂₄ alkylcarbonyl or C₁₋₂₄ alkylcarbonyl substituted one or more times by one or more NR′R′, halogen or ammonium salt; benzyl, benzoyl or benzyl or benzoyl substituted one or more times by one or more halogens, hydroxyl, C₁₋₁₂ alkyl, C₁₋₁₂ alkoxy or C₁₋₁₂ alkylcarboxy; or C₁₋₂₄ alkyl or C₁₋₂₄ alkylcarbonyl substituted by

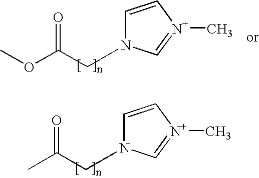

wherein n is a number from 1 to 12;
or R is a group

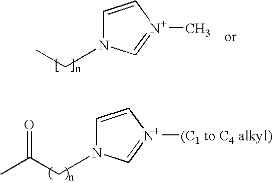

wherein n is a number from 1 to 12.

For example R is selected from C₁₋₂₄ alkyl and C₁₋₂₄ alkylcarbonyl which are substituted by at least one NR′R′ wherein each R′ is C₁₋₂₄alkyl or C₁₋₂₄alkylcarbonyl.

For example R is selected from C₁₋₂₄ alkyl and C₁₋₂₄ alkylcarbonyl which are substituted by at least one NR′R′ and at least one halogen, OR′ SO₃M, SO₃H, or a group of the formulae L-Ar, or;

for example, C₁₋₂₄ alkyl and C₁₋₂₄ alkylcarbonyl substituted by at least one NR′R′ and at least one halogen, OR′ or a group of the formulae

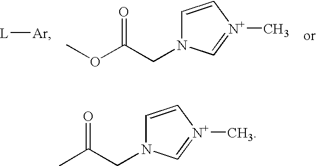

For example, R is selected from C₁₋₂₄ alkyl, C₁₋₂₄ alkylcarbonyl, C₃₋₂₄ alkenyl, and C₃₋₂₄ alkenylcarbonyl interrupted one or more times by one or more —O—, —N(R′)—, —CON(R′)—, —SO— or —SO₂—, and/or substituted one or more times by one or more group of the formulae

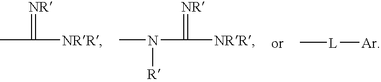

One particular embodiment of the invention relates to the polyglycerol anti-microbial agents, their preparation and formulations and methods of using them as anti-microbials which agents are hyperbranched polymers and dendrimers comprising in the backbone of the polymer the glycerol derived moieties

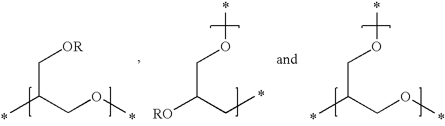

wherein the groups R are selected from
C₁₋₂₄ alkyl, C₃₋₂₄ alkenyl, C₁₋₂₄ alkylcarbonyl or C₃₋₂₄ alkenylcarbonyl which are interrupted one or more times by one or more —O—, —N(R′)—, —CON(R′)—, or said interrupted C₁₋₂₄ alkyl, C₃₋₂₄ alkenyl, C₁₋₂₄ alkylcarbonyl or C₃₋₂₄ alkenylcarbonyl substituted one or more times by one or more —OR′, —CONR′R′, —NR′R′, ammonium salt, group of the formulae

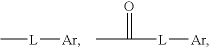

or group —Si(G)₃ wherein each G is independently hydroxyl, C₁₋₄ alkyl or C₁₋₄ alkoxy,
wherein R′, L and Ar are as defined above.

For example, the groups R are selected from C₁₋₂₄ alkyl, C₃₋₂₄ alkenyl or C₁₋₂₄ alkylcarbonyl which are interrupted one or more times by one or more —O— or —N(R′)—, or said interrupted C₁₋₂₄ alkyl, C₃₋₂₄ alkenyl or C₁₋₂₄ alkylcarbonyl substituted one or more times by one or more —OR′, —NR′R′ or ammonium salt, wherein R′ is H, C₁₋₂₄ alkyl, C₃₋₂₄ alkenyl, or C₁₋₂₄ alkylcarbonyl.

For Example, R is a group

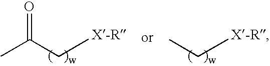

for example

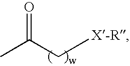

wherein X′ is —O—, —NH—, N(C₁₋₂₄ alkyl) or N(C₁₋₂₄ alkyl substituted by one or more hydroxy and/or C₁₋₁₂ alkoxy), R″ is C₁₋₂₄ alkyl or C₁₋₂₄ substituted by one or more hydroxy and/or C₁₋₁₂ alkoxy and w is a number from 1 though 12, for example, from 1 through 6.

For example X′ is —NH— or N(C₁₋₂₄ alkyl) and R″ is C₁₋₂₄ alkyl.

The polymer wherein, R is a group

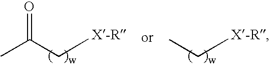

and X′ is amino is conveniently prepared by treating a hyperbranched glycerol polymer containing the moieties

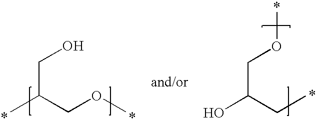

with a compound such as

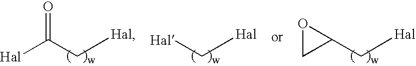

wherein each Hal or Hal′ is a halogen, for example chlorine or bromine, followed by reaction with an amine.

For example, novel compounds of the invention are the above described compounds wherein two different R groups are present, for example when a mixture of at least two R groups are present wherein at least two R groups are independently C₁₋₂₄ alkyl and/or C₁₋₂₄ alkylcarbonyl groups substituted by amino, alkoxy and/or hydroxyl groups as described above.

### EXAMPLES

The following non-limiting examples illustrate some aspects of the invention.

### Example 1

### Branched Glycerol Polymer

To propylene glycol (3.0 gms) in a 500 mL round bottomed flask is added and sodium methoxide (3.0 gms, 25 wt % in methanol) and methanol is removed by rotoevaporation. Glycidol is added via a dosing pump at a rate of 10 mL/hour at 90° C., the reaction mixture is then heated for an additional 4 hours after which time the polymer is dissolved in hot methanol. The resulting mixture in methanol is added to acetone and the polyglycerol polymer precipitated as brown syrup (104.1 gms).

### Example 2

To a solution of the polyglycerol from Example 1 (2.0 gms) in DMF (15.5 gms) cooled with an external ice/brine bath is added triethylamine (2.73 gms). 6-bromohexanoyl chloride is added dropwise over 10 minutes and the reaction is heated at 60° C. for 48 hours. The resulting suspension is filtered and concentrated to give as a yellow syrup (3.73 gms) a polyglycerol polymer containing the following substitution:

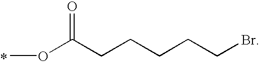

### Example 3

To the polyglycerol bromide from Example 2 is added dodecylamine (2.39 gms) and potassium hydroxide (0.72 gms) in ethanol (20 gms) and the mixture is stirred for 22 hours at 80° C., allowed to cool then filtered through Celite and concentrated to give as a yellow semisolid (4.0 gms) a polyglycerol polymer containing the following substitution:

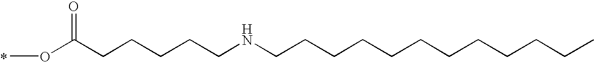

### Example 4

To a solution of polyglycerol from Example 1 (2.0 gms) in DMF (17 gms) is added triethylamine (5.4 gms) and the mixture cooled with an ice/brine bath. Acryloyl chloride is added dropwise over 20 minutes and the reaction mixture is allowed to warm to room temperature with stirring for 48 hours. The resulting suspension is filtered and concentrated to give as a yellow syrup a polyglycerol polymer containing the following substituent:

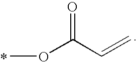

### Example 5

To the product of Example 4 is added dodecylamine (10.0 gms) in chloroform (29 gms) and the mixture stirred at room temperature for 48 hours. The reaction mixture is concentrated to give a yellow semisolid which is washed with ethyl acetate and filtered to give as an off-white solid (10.3 gms) a polyglycerol polymer containing the following substituent:

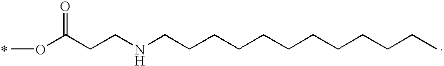

### Example 6

A mixture of a solution of the polyglycerol from Example 1 in DMF, benzyl bromide and potassium carbonate is stirred to prepare a polyglycerol polymer containing the following substituent:

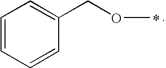

### Example 7

Following the procedure of example 2, to a solution of the polyglycerol from Example 1 in DMF cooled with an external ice/brine bath is added triethylamine then 1-chloroacetyl chloride is to prepare a polyglycerol polymer containing the following substitution:

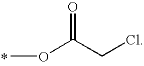

### Example 8

Following the procedure of example 3, to the polyglycerol bromide from Example 2 is added octylamine and potassium hydroxide in ethanol to yield a polyglycerol polymer containing the following substitution:

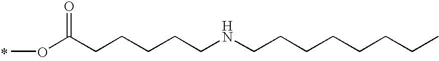

### Example 9

Following the procedure of example 3, to the polyglycerol bromide from Example 2 is added hexylamine and potassium hydroxide in ethanol to yield a polyglycerol polymer containing the following substitution:

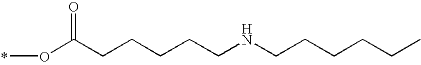

### Example 10

Following the procedure of example 3, to the polyglycerol bromide from Example 2 is added N,N-di-dodecylamine and potassium hydroxide in ethanol to yield a polyglycerol polymer containing the following substitution:

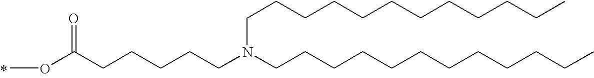

### Example 11

Following the procedure of example 3, to the polyglycerol bromide from Example 2 is added N-octylamine and potassium hydroxide in ethanol to yield a polyglycerol polymer containing the following substitution:

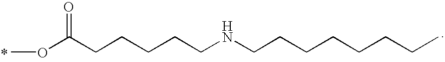

### Example 12

Following the procedure of example 3, to the polyglycerol bromide from Example 2 is added N-benzylamine and potassium hydroxide in ethanol to yield a polyglycerol polymer containing the following substitution:

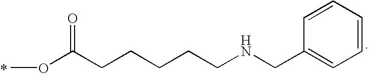

### Example 13

Following the procedure of example 3, to the polyglycerol bromide from Example 2 is added a mixture of N-octylamine and benzylamine and potassium hydroxide in ethanol to yield a polyglycerol polymer wherein different glycerol derived moieties containing one the following substituents are available:

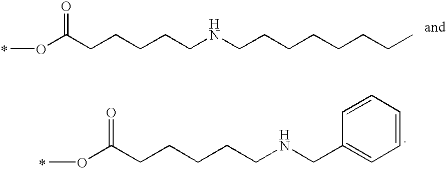

### Microbiological Activity:

The polymers from the Examples 1-10 are tested for activity against bacteria, e. coli, s. aureus; fungi, a. pull, p. funic, a. niger, adhesion of microbes or biofilm accumulation. All compounds are effective in at least one test; some are effective in more than one test.

Microbicidal activity is tested according to trivial modifications of the standard EN1040 test method. A bacterial suspension with a cell count of about 10⁷ cfu/m¹ is contacted with appropriate concentrations of the specific substances and the residual cell count is determined after incubation times of 5 and 30 min. at room temperature under continuous stirring. Staphylococcus aureus is tested as gram+ and Escherichia coli as gram-organism. The resulting cell count reduction is compared to a water control.

Fungicidal activity is tested according to trivial modifications of the standard EN12175 test method. A fungal spore suspension with a spore cell count of about 10⁶ cfu/m¹ is contacted with appropriate concentrations of the specific substances and the residual spore cell count is determined after incubation times of 30 and 60 min. at room temperature under continuous stirring. Penicillium funiculosum, Aspergillus niger and Aureobasidium pullulans are tested as important mold strains. The resulting cell count reduction is compared to a water control.

Biofilm inhibition is tested in a microplate based screening assay. Standard test specimen of polycarbonate are contacted with compound solutions in water or ethanol at a concentration of 0.5% for ½ hour for the compounds to form a film on the pin surface. The pins are then dried at room temperature under laminar flow. The coated pins are contacted with a bacterial inoculum of Staphylococcus aureus at a cell count of 10⁴-10⁵ cfu/ml in a microplate and a biofilm is allowed to form on the plastic surface over 24 hours. Loosely attached cells are then rinsed off in a couple of rinsing steps, then the biofilm on the surface is removed by ultrasonic treatment. The eluted cells are transferred into new microplates in Caso broth and growth is followed by measurement of optical density at 620 nm over 24 hours. The results are evaluated as growth curves of the eluted cells over 24 hours incubation time in comparison to the growth curve of untreated samples.

We claim:

1. An anti-microbial home or personal care formulation, plant protection formulation, natural or synthetic polymer composition, coating formulation or other material of construction composition comprising a branched polyglycerol anti-microbial polymer or copolymer comprising in the backbone of the polymer or copolymer glycerol derived moieties selected from 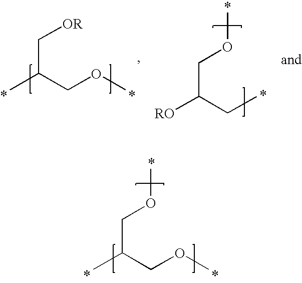
wherein R is independently H or a substituted or unsubstituted alkyl, alkenyl, alkanoyl, alkenoyl, aryl or heterocycle.

2. An anti-microbial formulation or composition according to claim 1, containing a polymer comprising a glycidol derived moiety wherein R is selected from H,
a) C₁₋₂₄ alkyl, C₃₋₂₄ alkenyl, C₁₋₂₄ alkylcarbonyl or C₃₋₂₄ alkenylcarbonyl which are uninterrupted or interrupted one or more times by one or more —O—, —N(R′)—, —CON(R′)—, —SO— or —SO₂—, and are unsubstituted or substituted one or more times by one or more C₃₋₆ cycloalkyl, —OR′, —COOR′, —COOM, —SO₃M, —SO₃H, phosphonic acid, halogen, —CONR′R′, —NR′R′, phosphonate salt, ammonium salt, group of the formulae 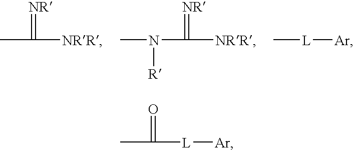
or group —Si(G)₃ wherein each G is independently hydroxyl, C₁₋₄ alkyl or C₁₋₄ alkoxy;
b) C₆₋₁₄ aromatic or C₁₋₉ saturated or unsaturated heterocycle which are unsubstituted or substituted one or more times by one or more groups R′, —OR′, —COOR′, —COOM, —SO₃M, —SO₃H, phosphonic acid, halogen, —CONR′R′, —NR′R′, phosphonate salt or ammonium salt,
c) a group of the formulae 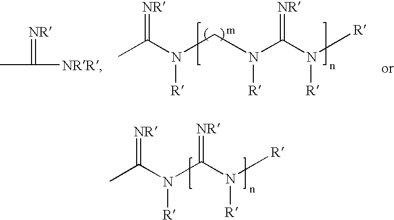
wherein m and n independently are a number from 1 to 12;
wherein each R′, independently of any other R′ is hydrogen;
a group 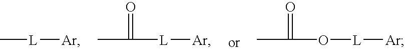
C₁₋₂₄ alkyl, C₃₋₂₄ alkenyl, C₃₋₆ cycloalkyl or C₁₋₂₄ alkylcarbonyl which are uninterrupted or interrupted one or more times by one or more oxygen atoms, sulfur atoms, carbonyl, —COO—, —CONH—, —NH—, —CON(C₁₋₂₄ alkyl)- or —N(C₁₋₂₄ alkyl)-,
which uninterrupted or interrupted alkyl, alkenyl, cycloalkyl or alkylcarbonyl are unsubstituted or substituted one or more times by one or more groups selected from halogen, —OH, C₂₋₂₄alkylcarbonyl, C₁₋₂₄alkoxy, C₂₋₂₄alkylcarboxy, —COOM, —CONH₂, —CON(H)(C₁₋₂₄ alkyl), —CON(C₁₋₂₄ alkyl)₂, —NH₂, —N(H)(C₁₋₂₄ alkyl), —N(C₁₋₂₄ alkyl)₂, —SO₃M, purine, pyridine, pyrimidine, triazine, imidazole, wherein each purine, pyridine, pyrimidine, triazine or imidazole is unsubstituted or substituted by one or more C₁₋₁₂ alkyl and wherein the purine, pyridine, pyrimidine, triazine or imidazole is neutral or ionically charged, phenyl, phenyl substituted one or more times by one or more C₁₋₈ alkyl, naphthyl, naphthyl substituted one or more times by one or more C₁₋₈ alkyl, amidine, guanidine, ammonium salt, phosphonic acid, phosphonate salt and a group 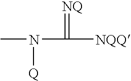
wherein each Q or Q′ is independently hydrogen, C₁₋₁₂alkyl, phenyl or benzyl;
or when two R′ are attached to a nitrogen atom they may form, together with the nitrogen atom to which they are attached, form a 5-, 6- or 7-membered ring which is uninterrupted or interrupted by —O—, —NH— or —N(C₁₋₁₂ alkyl)-;
L is a direct bond, C₁₋₁₂ alkylene which is uninterrupted or interrupted by one or more oxygen atoms, —NH—, —N(C₁₋₁₂ alkyl) or phenylene and/or unsubstituted or substituted one or more times by one or more —OH, C₁₋₈ alkyl, C₁₋₂₄ alkoxy, C₂₋₂₄alkylcarboxy, —NH₂, —N(H)(C₁₋₈ alkyl), —N(C₁₋₈ alkyl)₂ or ammonium salt:
Ar is C₆₋₁₀ aromatic or C₁₋₉ saturated or unsaturated heterocycle which C₆₋₁₀ aromatic or C₁₋₉ saturated are unsubstituted or substituted one or more times by one or more halogen, —OH, C₁₋₂₄ alkoxy, C₂₋₂₄ alkylcarboxy, —COOQ″, —CONH₂, —CON(H)(C₁₋₈ alkyl), —CON(C₁₋₈alkyl)₂, —NH₂, —N(H)(C₁₋₈alkyl), —N(C₁₋₈alkyl)₂, —SO₃M, SO₃H, ammonium salt, phosphonic acid, phosphonate salt, C₁₋₂₄ alkyl, C₁₋₂₄ alkyl or C₂₋₂₄ alkylcarboxy which is substituted one or more times by one or more groups selected from halogen, phenyl, phenyl substituted one or more times by one or more C₁₋₈ alkyl, naphthyl, purine, pyridine, pyrimidine, triazine and imidazole, wherein the purine, pyridine, pyrimidine, triazine or imidazole is unsubstituted or substituted by one or more C₁₋₁₂ alkyl and wherein the purine, pyridine, pyrimidine, triazine or imidazole is neutral or ionically charged;
wherein Q″ is hydrogen, C₁₋₂₄ alkyl, metal cation, ammonium salt, glycol ether, phenyl or benzyl, or phenyl or benzyl substituted one or more times by one or more halogen, hydroxy, C₁₋₂₄ alkoxy or C₁₋₁₂ alkyl,
and M is a metal cation or an ammonium cation.

3. An anti-microbial formulation or composition according to claim 2, containing a polymer comprising a glycidol derived moiety wherein R is selected from H, C₁₋₂₄ alkyl, C₃₋₂₄ alkenyl, C₁₋₂₄ alkylcarbonyl or C₃₋₂₄ alkenylcarbonyl which are uninterrupted or interrupted one or more times by one or more —O—, —N(R′)—, —CON(R′)—, —SO— or —SO₂—, and are unsubstituted or substituted one or more times by one or more C₃₋₆ cycloalkyl, —OR′, —COOR′, —COOM, —SO₃M, —SO₃H, phosphonic acid, halogen, —CONR′R′, —NR′R′, phosphonate salt, ammonium salt, group of the formulae 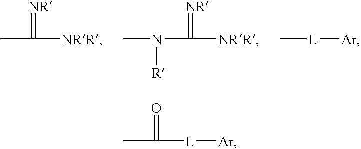
or group —Si(G)₃.

4. An anti-microbial formulation or composition according to claim 2, containing a polymer comprising a glycidol derived moiety wherein R is independently selected C₆₋₁₄ aromatic and C₁₋₉ saturated or unsaturated heterocycle which are unsubstituted or substituted one or more times by one or more groups R′, —OR′, —COOR′, —COOM, —SO₃M, —SO₃H, phosphonic acid, halogen, —CONR′R′, —NR′R′, phosphonate salt or ammonium salt.

5. An anti-microbial formulation or composition according to claim 4, containing a polymer comprising a glycidol derived moiety wherein R is selected a group 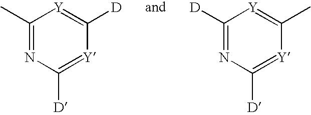
wherein Y and Y′ are independently N, C—R′, C—OR′ or C—NR′R′ and D and D′ are independently R′, —OR′ or —NR′R′.

6. An anti-microbial formulation or composition according to claim 2, containing a polymer comprising a glycidol derived moiety wherein R is selected from the formulae 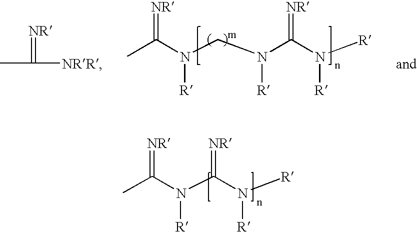
wherein m and n independently are 1, 2, 3, 4, 5 or 6.

7. An anti-microbial formulation or composition according to claim 2, containing a polymer comprising a glycidol derived moiety wherein R is selected from C₁₋₂₄ alkyl and C₁₋₂₄ alkylcarbonyl which are uninterrupted or interrupted one or more times by —O—, —N(R′)—, —CON(R′)—, and are unsubstituted or substituted by one or more —NR′R′, halogen, ammonium salt,

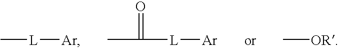

8. An anti-microbial formulation or composition according to claim 2, containing a polymer comprising a glycidol derived moiety wherein R is selected from C₁₋₂₄ alkyl and C₁₋₂₄ alkylcarbonyl which are uninterrupted or interrupted one or more times by —O—, and substituted by one or more —NR′R′, halogen, ammonium salt, -L-Ar, 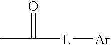
or —OR′,
wherein
R′ is hydrogen; -L-Ar, 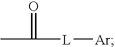
C₁₋₂₄ alkyl or C₁₋₂₄ alkylcarbonyl which alkyl or alkylcarbonyl are uninterrupted or interrupted one or more times by one or more oxygen atoms, —COO—, —CONH—, —NH—, —CON(C₁₋₂₄ alkyl)- or —N(C₁₋₂₄ alkyl)- and which uninterrupted or interrupted alkyl or alkylcarbonyl are unsubstituted or substituted one or more times by one or more groups selected from halogen, —OH, C₂₋₂₄alkylcarbonyl, C₁₋₂₄alkoxy, C₂₋₂₄alkylcarboxy, —COOM, —CONH₂, —CON(H)(C₁₋₂₄ alkyl), —CON(C₁₋₂₄ alkyl)₂, —NH₂, —N(H)(C₁₋₂₄ alkyl), —N(C₁₋₂₄ alkyl)₂, purine, pyridine, pyrimidine, triazine, imidazole, wherein each purine, pyridine, pyrimidine, triazine or imidazole is unsubstituted or substituted by one or more C₁₋₁₂ alkyl and wherein the purine, pyridine, pyrimidine, triazine or imidazole is neutral or ionically charged, phenyl, phenyl substituted one or more times by one or more C₁₋₈ alkyl, naphthyl, naphthyl substituted one or more times by one or more C₁₋₈ alkyl and ammonium salt.

9. An anti-microbial formulation or composition according to claim 2, containing a polymer comprising a glycidol derived moiety wherein R is selected from C₁₋₂₄ alkyl, C₁₋₂₄ alkyl substituted one or more times by one or more NR′R′, halogen or ammonium salt; C₁₋₂₄ alkylcarbonyl, C₁₋₂₄ alkylcarbonyl substituted one or more times by one or more NR′R′, halogen or ammonium salt; benzyl, benzoyl which benzyl or benzoyl may be substituted one or more times by one or more halogens, hydroxyl, C₁₋₁₂ alkyl, C₁₋₁₂ alkoxy or C₁₋₁₂ alkylcarboxy; and C₁₋₂₄ alkyl or C₁₋₂₄ alkylcarbonyl substituted by

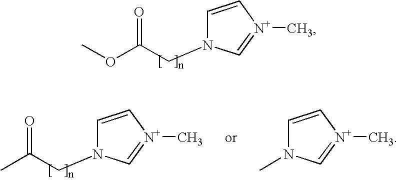

10. An anti-microbial formulation or composition according to claim 2, containing a polymer comprising a glycidol derived moiety wherein R is selected from C₁₋₂₄ alkyl or C₁₋₂₄ alkylcarbonyl which are substituted by at least one NR′R′ wherein each R′ is C₁₋₂₄ alkyl or C₁₋₂₄ alkylcarbonyl;
C₁₋₂₄ alkyl or C₁₋₂₄ alkylcarbonyl which are substituted by at least one NR′R′ and at least one halogen, OR′ SO₃M, SO₃H, or a group of the formulae L-Ar and
C₁₋₂₄ alkyl or C₁₋₂₄ alkylcarbonyl substituted by at least one NR′R′ and at least one halogen, OR′ or a group of the formulae

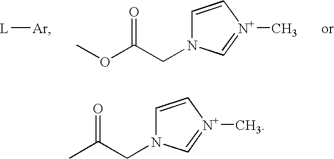

11. An anti-microbial formulation or composition according to claim 2, containing a polymer comprising at least two glycidol derived moieties with different R groups.

12. A method for protecting plastics, coatings, other materials of construction, home or personal care formulations, industrial formulations, or technical process against the action of microbes which comprises adding an effective amount of a polymer or copolymer of claim 1 to the plastics, coatings, other materials of construction, home or personal care formulations, industrial formulations, or during the technical process.

13. A method for protecting skin, mucosa, integumentary appendages, plants, seeds and fruit against the action of microbes which comprises applying a preparation comprising an effective amount of a polymer or copolymer of claim 1.

14. A method for protecting paper, wood, leather or textile materials against the action of microbes comprising incorporating into or applying onto an effective amount of a polymer or copolymer of claim 1.

15. A personal care preparation, oral hygiene formulation or washing and cleaning formulation comprising a polymer or copolymer of claim 1.

16. A personal care preparation, oral hygiene formulation or washing and cleaning formulation according to claim 15 which is an anti-dandruff composition.

17. A composition comprising a polymer or copolymer of claim 2 and another natural or synthetic polymer.

18. A composition comprising more than one polymer or copolymer of claim 1.

19. A composition according to claim 17 which is a woven or non woven textile, paper product, coating composition or plastic article.

20. A method for cleaning and disinfecting hard surfaces which comprises applying a preparation comprising an effective amount of a polymer or copolymer of claim 1.

21. A method for preventing bio-fouling of an article comprising incorporating the antimicrobial polymers or co-polymers of claim 1 into the article or surface of the article or by applying the antimicrobial polymers or co-polymers to the surfaces either directly or as part of a coating or film.

22. A hyperbranched polymer or dendrimers comprising in the backbone of the polymer a glycerol derived moiety selected from 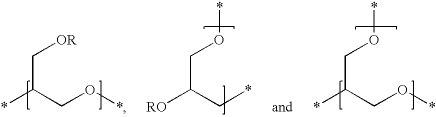
wherein the group R is selected from
a) C₁₋₂₄ alkyl, C₃₋₂₄ alkenyl, C₁₋₂₄ alkylcarbonyl or C₃₋₂₄ alkenylcarbonyl which are interrupted one or more times by one or more —O—, —N(R′)—, —CON(R′)—, —SO— or —SO₂—, and/or substituted one or more times by one or more —CONR′R′, —NR′R′, ammonium salt, group of the formulae 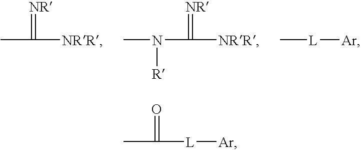
or group —Si(G)₃ wherein each G is independently hydroxyl, C₁₋₄ alkyl or C₁₋₄ alkoxy;
b) C₁₋₉ saturated or unsaturated heterocycle which are unsubstituted or substituted one or more times by one or more groups R′, —OR′, —COOR′, —COOM, —SO₃M, —SO₃H, phosphonic acid, halogen, —CONR′R′, —NR′R′, phosphonate salt or ammonium salt, including a heterocycle of the formulae 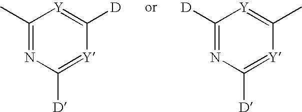
wherein Y and Y′ are independently N, C—R′, C—OR′ or C—NR′R′ and D and D′ are independently R′, —OR′ or —NR′R′; and
c) group of the formulae 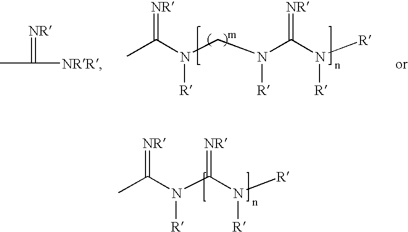
wherein m and n independently are 1, 2, 3, 4, 5 or 6;
wherein each R′, independently of any other R′ is hydrogen; 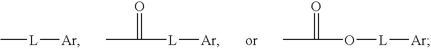
a group
C₁₋₂₄ alkyl, C₃₋₂₄ alkenyl, C₃₋₆ cycloalkyl or C₁₋₂₄ alkylcarbonyl which are uninterrupted or interrupted one or more times by one or more oxygen atoms, sulfur atoms, carbonyl, —COO—, —CONH—, —NH—, —CON(C₁₋₂₄ alkyl)- or —N(C₁₋₂₄ alkyl)-,
which uninterrupted or interrupted alkyl, alkenyl, cycloalkyl or alkylcarbonyl are unsubstituted or substituted one or more times by one or more groups selected from halogen, —OH, C₂₋₂₄alkylcarbonyl, C₁₋₂₄alkoxy, C₂₋₂₄alkylcarboxy, —COOM, —CONH₂, —CON(H)(C₁₋₂₄ alkyl), —CON(C₁₋₂₄ alkyl)₂, —NH₂, —N(H)(C₁₋₂₄ alkyl), —N(C₁₋₂₄ alkyl)₂, —SO₃M, purine, pyridine, pyrimidine, triazine, imidazole, wherein each purine, pyridine, pyrimidine, triazine or imidazole is unsubstituted or substituted by one or more C₁₋₁₂ alkyl and wherein the purine, pyridine, pyrimidine, triazine or imidazole is neutral or ionically charged, phenyl, phenyl substituted one or more times by one or more C₁₋₈ alkyl, naphthyl, naphthyl substituted one or more times by one or more C₁₋₈ alkyl, amidine, guanidine, ammonium salt, phosphonic acid, phosphonate salt and a group 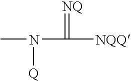
wherein each Q or Q′ is independently hydrogen, C₁₋₁₂alkyl, phenyl or benzyl;
or
when two R′ are attached to a nitrogen atom they may form, together with the nitrogen atom to which they are attached, form a 5-, 6- or 7-membered ring which is uninterrupted or interrupted by —O—, —NH— or —N(C₁₋₁₂ alkyl)-;
L is a direct bond, C₁₋₁₂ alkylene which is uninterrupted or interrupted by one or more oxygen atoms, —NH—, —N(C₁₋₁₂ alkyl) or phenylene and/or unsubstituted or substituted one or more times by one or more —OH, C₁₋₈ alkyl, C₁₋₂₄ alkoxy, C₂₋₂₄alkylcarboxy, —NH₂, —N(H)(C₁₋₈ alkyl), —N(C₁₋₈ alkyl)₂ or ammonium salt:
Ar is C₆₋₁₀ aromatic or C₁₋₉ saturated or unsaturated heterocycle which C₆₋₁₀ aromatic or C₁₋₉ saturated are unsubstituted or substituted one or more times by one or more halogen, —OH, C₁₋₂₄ alkoxy, C₂₋₂₄ alkylcarboxy, —COOQ″, —CONH₂, —CON(H)(C₁₋₈ alkyl), —CON(C₁₋₈alkyl)₂, —NH₂, —N(H)(C₁₋₈alkyl), —N(C₁₋₈alkyl)₂, —SO₃M, SO₃H, ammonium salt, phosphonic acid, phosphonate salt, C₁₋₂₄ alkyl, C₁₋₂₄ alkyl or C₂₋₂₄ alkylcarboxy which is substituted one or more times by one or more groups selected from halogen, phenyl, phenyl substituted one or more times by one or more C₁₋₈ alkyl, naphthyl, purine, pyridine, pyrimidine, triazine and imidazole, wherein the purine, pyridine, pyrimidine, triazine or imidazole is unsubstituted or substituted by one or more C₁₋₁₂ alkyl and wherein the purine, pyridine, pyrimidine, triazine or imidazole is neutral or ionically charged;
wherein Q″ is hydrogen, C₁₋₂₄ alkyl, metal cation, ammonium salt, glycol ether, phenyl or benzyl, or phenyl or benzyl substituted one or more times by one or more halogen, hydroxy, C₁₋₂₄ alkoxy or C₁₋₁₂ alkyl,
M is a metal cation or an ammonium cation.
